(19) United States
(12) Patent Application Publication
(10) Pub. No.: US 2025/0115560 A1
(43) Pub. Date: Apr. 10, 2025

(54) A NEW PROCESS OF SAFLUFENACIL PRODUCTION USING NOVEL INTERMEDIATES

(71) Applicant: ADAMA AGAN LTD., Ashdod (IL)

(72) Inventors: Michael GRABARNICK, Meitar (IL); Ashok Kumar JHA, Telangana (IN); Revanappa Vasantrao GALGE, Maharashtra (IN)

(73) Assignee: ADAMA AGAN LTD, Ashdod (IL)

(21) Appl. No.: 18/294,602

(22) PCT Filed: Aug. 9, 2022
(86) PCT No.: PCT/IL2022/050870

§ 371 Date: Feb. 2, 2024

(30) Foreign Application Priority Data
Aug. 9, 2021 (IN) 202111035923

(51) Int. Cl. C07D 239/557

(52) U.S. Cl. CPC C07D 239/557

(57) ABSTRACT

The invention relates to a novel and efficient saflufenacil synthesis and the preparation of novel key intermediates in the process of Saflufenacil synthesis. The process includes preparing compounds of the Formula I wherein R₁i is a hydrogen, C1-12 straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; R₂ is a hydrogen or methyl.

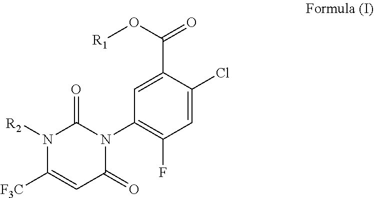

### TECHNICAL FIELD OF THE INVENTION

The present invention relates to a novel and an efficient process for preparation of Saflufenacil using novel intermediates.

### BACKGROUND OF THE INVENTION

Saflufenacil, having the chemical name 2-chloro-5-[3,6-dihydro-3-methyl-2,6-dioxo-4-(trifluoromethyl)-1(2H-pyrimidinyl]-4-fluoro-N-[[methyl(1-methylethyl)amino]sulfonyl]benzamide, has the following structural Formula (1):

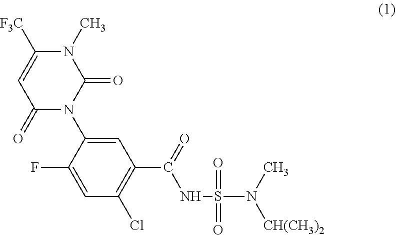 [SMILES: CC(C)N(C)S(=O)(=O)NC(=O)c1cc(-n2c(=O)cc(C(F)(F)F)n(C)c2=O)c(F)cc1Cl]

Saflufenacil belongs to the pyrimidindione and/or phenyluracil chemical groups and is used as an herbicide, in particular as a foliar contact and residual broad-leaved weed herbicide. It is absorbed by foliage and roots with translocation in the apoplast and limited movement in the phloem. Saflufenacil is applied to foliage and is used for residual control of broad-leaved weeds, including glyphosate-and ALS-resistant biotypes. Saflufenacil is an inhibitor of protoporphyrinogen oxidase and is applied pre-emergence in corn and sorghum, at 50-125 g/ha; and is applied pre-plant for rapid foliar burn-down in soybeans, cereals, cotton, legumes, and post-directed in tree fruit and nuts, at 18-25 g/ha.

Saflufenacil is disclosed in WO 2001/083459. Further different steps of the processes for its preparation are disclosed in WO 2003/097589, WO 2005/054208 and WO 2006/010474 and the earlier international application PCT/EP2006/062414. Furthermore, the two crystalline modifications of Saflufenacil, known in the art, Saflufenacil form II and crystalline form of Saflufenacil hydrate, disclosed in WO 2008/043835 and WO 2008/043836.

All known ways of synthesis include either expensive and not commercially available reagents like 2-dimethylamino-4-(trifluoromethyl)-6H-1,3-oxazin-6-one or/and demand introduction of the a sulfamide group in the molecule on the early steps of the synthesis and accordingly, enlarge the production cost.

Therefore, there is a need in the art for a new and efficient way of Saflufenacil synthesis.

### SUMMARY OF THE INVENTION

The present invention provides a process for preparing compounds of the Formula I:

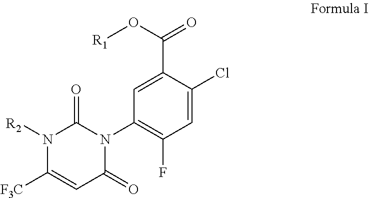

wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl;
the process comprising the following steps:
a) a reaction of the aniline moiety of the compound of Formula II:

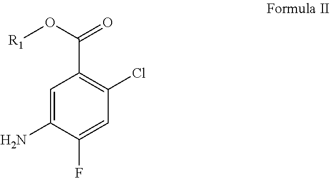

wherein R₁ is hydrogen with a carbonyl precursor, wherein the carbonyl precursor is ethyl 4,4,4-trifluoroacetoacetate, to obtain the compound of Formula II′:

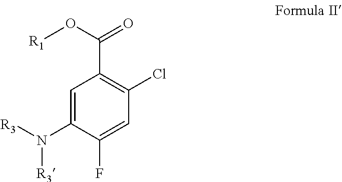

wherein R₁ is hydrogen;
R₃ is hydrogen;
R₃′ is 4,4,4-trifluoroacetoacetate; and
b) a reaction of the resulting compound of Formula II′ with a cyclization reagent which is KOCN in AcOH;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is phosgene, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ and R₃′ together are carbonyl; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is ethyl chloroformate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl formate; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents with a carbonyl precursor, wherein the carbonyl precursor is ethyl 4,4,4-trifluoroacetoacetate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl 4,4,4-trifluoroacetoacetate; and
b) a reaction of the resulting compound of Formula II′ with a cyclization reagent which is KOCN in AcOH.

Further, the present invention provides a compound of the general Formula I:

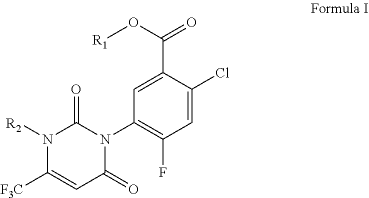

wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl.

Still further the present invention provides a N-[Methyl(isopropyl)aminosulfonyl][2-chloro-4-fluoro-5-(3-methylureido)benzamide.

Still further the present invention provides a 4-fluoro-N-(N-isopropyl-N-methylsulfamoyl)-2-methoxy-5-(3-methyl-2,6-dioxo-4-(trifluoromethyl)-3,6-dihydropyrimidin-1(2H)-yl)benzamide.

Still further the present invention provides a 3-(5-[N-Diethylaminosulfonylaminocarbonyl]-4-chloro-2-fluorophenyl)-2,4-dioxo-1-methyl-6-(trifluoromethyl)-1,2,3,4-tetrahydropyrimidine.

Still further the present invention provides a 3-[5-(N-Methyl[N-methyl(isopropyl)aminosulfonyl]aminocarbonyl)-4-chloro-2-fluorophenyl]-1-methyl-2,4-dioxo-6-(trifluoromethyl)-1,2,3,4-tetrahydropyrimidine.

Still further the present invention provides a 3-(5-[N-Methyl (isopropyl)aminosulfonylaminocarbonyl]-4-chloro-2-fluorophenyl)-2-methoxy-4-oxo-6-(trifluoromethyl)-3,4-dihydropyrimidine.

### DETAILED DESCRIPTION OF THE INVENTION

The present invention includes novel critical intermediates (e.g. compounds of the general Formula I) and a method to produce thereof in an efficient and cost effective Saflufenacil synthesis.

The present invention, in large, includes a process for preparing compounds of the Formula I:

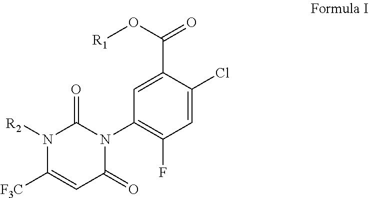

wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl;
said process comprises two steps:
a) formation of the backbone of the compound of Formula I by a reaction of the aniline moiety of the compound of Formula II:

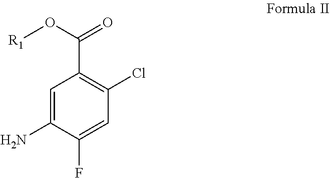

wherein R₁ is as defined for the compound of Formula I, with a carbonyl precursor, to obtain the compound of Formula II′:

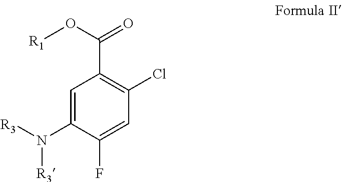

and
b) a reaction of the resulting compound of Formula II′ with a cyclization reagent which is KOCN in AcOH; followed by a cyclization reaction if needed;
wherein the substituents are dependent on the compound of Formula II and the carbonyl precursor of step a.

The current invention can utilize a range of carbonyl precursors which comprises: phosgene, ethyl 4,4,4-trifluoro-3-oxobutanoate, ethyl chloroformate.

Using intermediate of Formula I makes synthesis of Saflufenacil both more cost effective and reduces amount of impurities in the final product.

Cost improvement is based on introduction of the second expensive intermediate N-isopropyl-N-methylsulfamide on the last steps of the synthesis and accordingly, yield improvement on this compound and cost reduction.

Using intermediate of Formula I with R₂ is methyl prevents methylation of compound

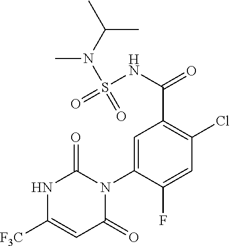 [SMILES: CC(C)N(C)S(=O)(=O)NC(=O)c1cc(-n2c(=O)cc(C(F)(F)F)[nH]c2=O)c(F)cc1Cl]

to Saflufenacil and so, make not possible production of the dimethyl impurity

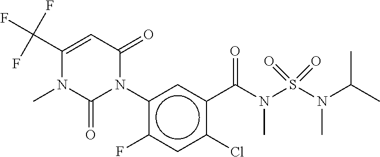

that is the main impurity in Saflufenacil synthesis, purifying from which is a challenging task.

Also, an efficient synthesis with high yields of Saflufenacil has not yet been reported. Therefore, there is an increasing need for an inexpensive, high yielding and efficient synthetic pathway towards Saflufenacil.

In one aspect of the present invention is a process for preparing compounds of the Formula I:

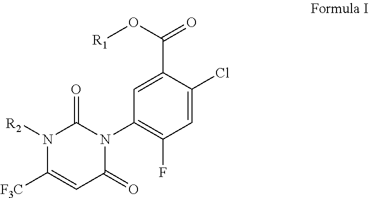

wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl;
the process comprising the following steps:
a) a reaction of the aniline moiety of the compound of Formula II:

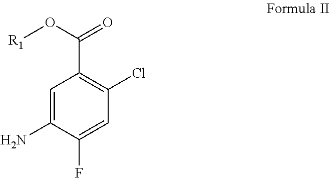

wherein R₁ is hydrogen with a carbonyl precursor, wherein the carbonyl precursor is ethyl 4,4,4-trifluoroacetoacetate, to obtain the compound of Formula II′:

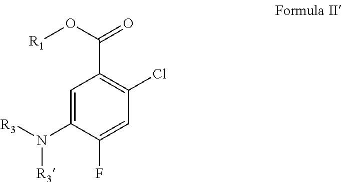

wherein R₁ is hydrogen;
R₃ is hydrogen;
R₃′ is 4,4,4-trifluoroacetoacetate; and
b) a reaction of the resulting compound of Formula II′ with a cyclization reagent which is KOCN in AcOH;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is phosgene, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ and R₃′ together are carbonyl; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is ethyl chloroformate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl formate; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents with a carbonyl precursor, wherein the carbonyl precursor is ethyl 4,4,4-trifluoroacetoacetate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl 4,4,4-trifluoroacetoacetate; and
b) a reaction of the resulting compound of Formula II′ with a cyclization reagent which is KOCN in AcOH.

In another aspect of the present invention is a process, wherein the carbonyl precursor comprises: phosgene, ethyl 4,4,4-trifluoro-3-oxobutanoate, ethyl chloroformate.

In yet another aspect of the present invention is a process, wherein a cyclization reagent comprises: potassium isocyanate in acetic acid, alkyl 3-amino-4,4,4-trifluorobut-2-enoate or alkyl 3-methylamino-4,4,4-trifluorobut-2-enoate.

In another aspect of the present invention is a process, wherein the process is carried out in an aprotic organic solvent selected from a group consisting of: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

In another aspect of the present invention is a process, wherein the process of step a) is carried out at a temperature of 0° C. to 150° C.

In another aspect of the present invention is a process, wherein the process of step b) is carried out at a temperature of 20° C. to 100° C.

In another aspect of the present invention is a process, comprising a step of a hydrolysis reaction of the compounds of Formula I where R₁ is not hydrogen using an acidic or basic catalysis, to obtain a compound of Formula III:

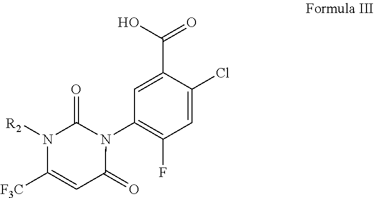

wherein
R₂ is a hydrogen or methyl.

In another aspect of the present invention is a process, comprises the step of condensation reaction of an acid of Formula III

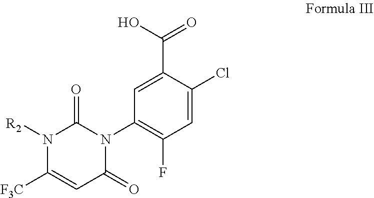

wherein
R₂ is a hydrogen or methyl; and a compound of Formula 2,

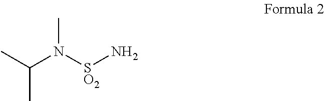

using a coupling reagent, to obtain the compound of Formula IV:

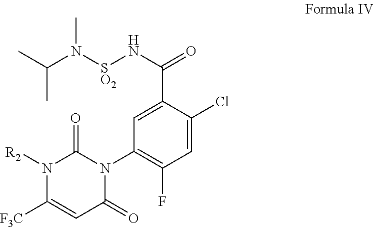

In another aspect of the present invention is a process, wherein the coupling reagent is selected from a list comprising: halogenated reagents like oxalyl chloride, thionyl chloride, phosgene, Vilsmeier reagents, CDI, carbon diimides, HBTU.

In another aspect of the present invention is a process, wherein the reaction is carried out in a solvent selected from a group comprising: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

In another aspect of the present invention is a process, wherein reaction is carried out in a temperature of 0° C. to 100° C.

It was surprisingly discovered that the above-mentioned condensation reaction is more efficient, reproducible and with the use of recyclable chemicals, thus leading to green chemistry and production.

In another aspect of the present invention is a process, comprises an additional step of methylation reaction on the compound of the Formula IV, when R₂ is hydrogen, using a methylation reagent, to obtain Saflufenacil:

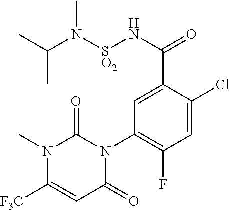 [SMILES: CC(C)N(C)S(=O)(=O)NC(=O)c1cc(-n2c(=O)cc(C(F)(F)F)n(C)c2=O)c(F)cc1Cl]

### Saflufenacil.

In another aspect of the present invention is a process, wherein the methylation reagent is selected from a list comprising: dimethyl sulfate, methyl bromide or methyl iodide

In another aspect of the present invention is a process, wherein the reaction is carried out in a solvent selected from a group comprising: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

In another aspect of the present invention is a process, wherein reaction is carried out in a temperature of 0° C. to 100° C.

The present invention includes a process of preparation of a compound of the general Formula II:

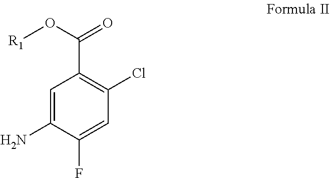

wherein the compound of the general Formula II is prepared by the following steps: (i) nitration reaction of the compound of 2-chloro-4-fluorobenzoic acid,

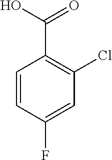

using nitration reagents system of sulfuric acid or oleum and nitric acid, to obtain 2-chloro-4-fluoro-5-nitrobenzoic acid:

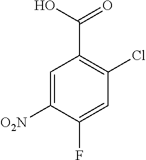 [SMILES: O=C(O)c1cc([N+](=O)[O-])c(F)cc1Cl]

followed by
(ii) alkylation reaction of 2-chloro-4-fluoro-5-nitrobenzoic acid of step (i) using an alkylation reagent, to obtain the compound of Formula VI:

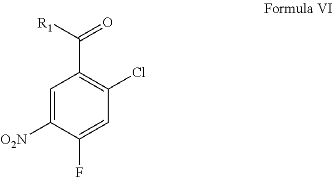

wherein R₁ is as defined in the previous claims; and
(iii) reduction reaction of the compound of Formula VI, using reduction reagent system, to obtain the compound of the general Formula II

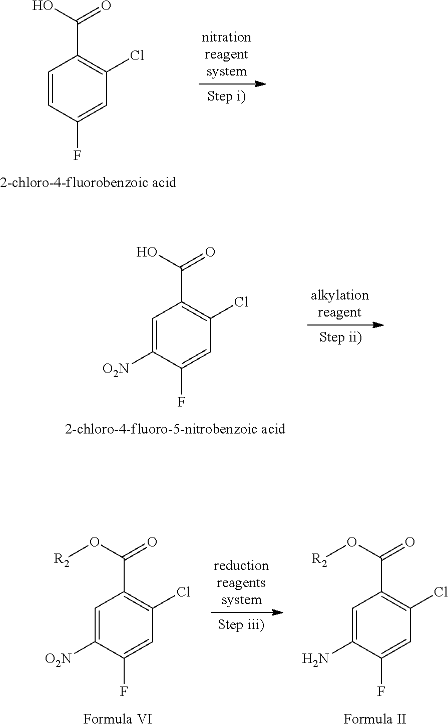

It was surprisingly discovered, that steps i) and ii) are interchangeable.

In one aspect of the present invention is a compound of the general Formula I:

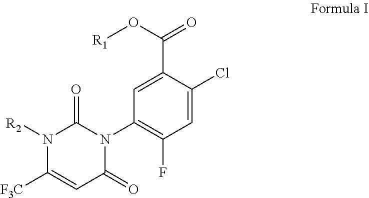

wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl.

It was surprisingly discovered that this new and efficient process, includes the creation of several novel compounds.

In one aspect of the present invention, is a N-[Methyl(isopropyl)aminosulfonyl][2-chloro-4-fluoro-5-(3-methylureido)benzamide:

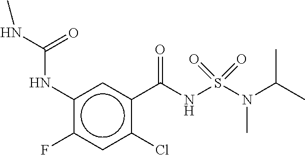

The N-[Methyl(isopropyl)aminosulfonyl][2-chloro-4-fluoro-5-(3-methylureido)benzamide is produced as a result of hydrolysis of Saflufenacil during purification process according to the scheme:

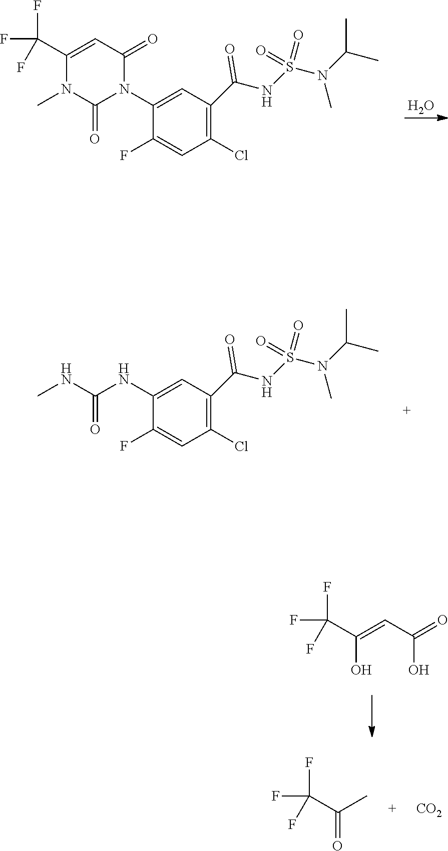

In one aspect of the present invention, is a 4-fluoro-N-(N-isopropyl-N-methylsulfamoyl)-2-methoxy-5-(3-methyl-2,6-dioxo-4-(trifluoromethyl)-3,6-dihydropyrimidin-1(2H)-yl)benzamide:

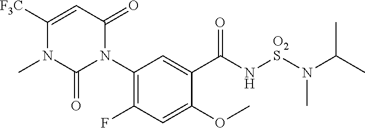 [SMILES: COc1cc(F)c(-n2c(=O)cc(C(F)(F)F)n(C)c2=O)cc1C(=O)NS(=O)(=O)N(C)C(C)C]

4-fluoro-N-(N-isopropyl-N-methylsulfamoyl)-2-methoxy-5-(3-methyl-2,6-dioxo-4-(trifluoromethyl)-3,6-dihydropyrimidin-1(2H)-yl)benzamide is produced from starting material of the general Formula IV wherein R₂ is methyl during sodium salt of Saflufenacil preparation in reaction with sodium methoxide according to the scheme:

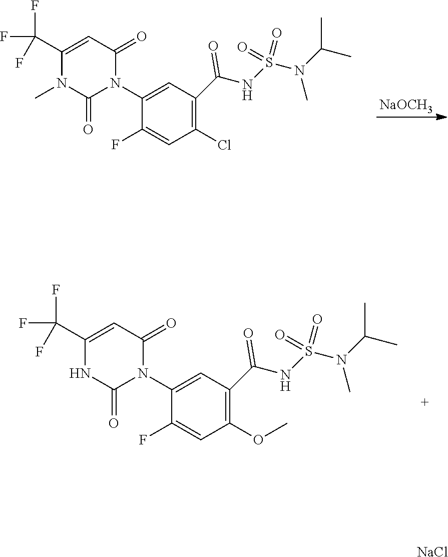

In one aspect of the present invention, is a 3-(5-[N-Diethylaminosulfonylaminocarbonyl]-4-chloro-2-fluorophenyl)-2,4-dioxo-1-methyl-6-(trifluoromethyl)-1,2,3,4-tetrahydropyrimidine:

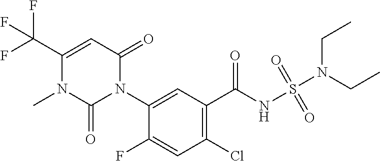 [SMILES: CCN(CC)S(=O)(=O)NC(=O)c1cc(-n2c(=O)cc(C(F)(F)F)n(C)c2=O)c(F)cc1Cl]

3-(5-[N-Diethylaminosulfonylaminocarbonyl]-4-chloro-2-fluorophenyl)-2,4-dioxo-1-methyl-6-(trifluoromethyl)-1,2,3,4-tetrahydropyrimidine formed as impurity along whole way of sulfone amide synthesis and final step of the compound of the general Formula IV. Triethylamine that is used as a base on the first step of sulfone amide production, reacts with sulfuryl chloride producing chain of the isomers according to the scheme:

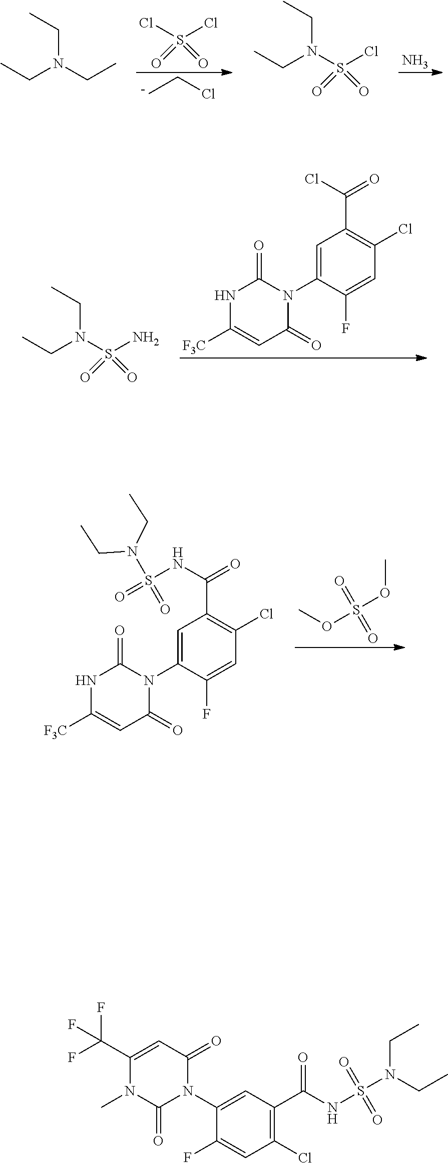

In one aspect of the present invention, is a 3-[5-(N-Methyl[N-methyl (isopropyl)aminosulfonyl]aminocarbonyl)-4-chloro-2-fluorophenyl]-1-methyl-2,4-dioxo-6-(trifluoromethyl)-1,2,3,4-tetrahydropyrimidine:

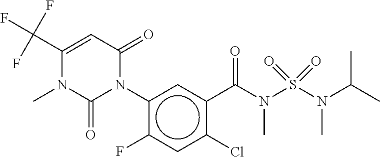

3-[5-(N-Methyl[N-methyl(isopropyl)aminosulfonyl]aminocarbonyl)-4-chloro-2-fluorophenyl]-1-methyl-2,4-dioxo-6-(trifluoromethyl)-1,2,3,4-tetrahydropyrimidine is produced in reaction of the compound of the general Formula IV wherein R₂ is hydrogen with excess of dimethylsulfate during Saflufenacil synthesis according to the scheme:

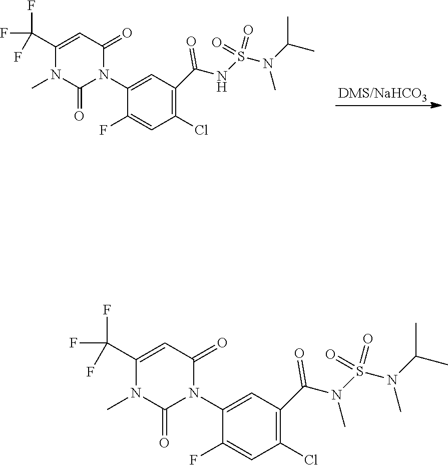

In one aspect of the present invention, is a 3-(5-[N-Methyl(isopropyl)aminosulfonylaminocarbonyl]-4-chloro-2-fluorophenyl)-2-methoxy-4-oxo-6-(trifluoromethyl)-3,4-dihydropyrimidine:

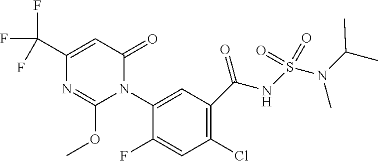 [SMILES: COc1nc(C(F)(F)F)cc(=O)n1-c1cc(C(=O)NS(=O)(=O)N(C)C(C)C)c(Cl)cc1F]

3-(5-[N-Methyl(isopropyl)aminosulfonylaminocarbonyl]-4-chloro-2-fluorophenyl)-2-methoxy-4-oxo-6-(trifluoromethyl)-3,4-dihydropyrimidine is produced as a result of O-methylation instead of N-methylation during Saflufenacil synthesis according to the scheme:

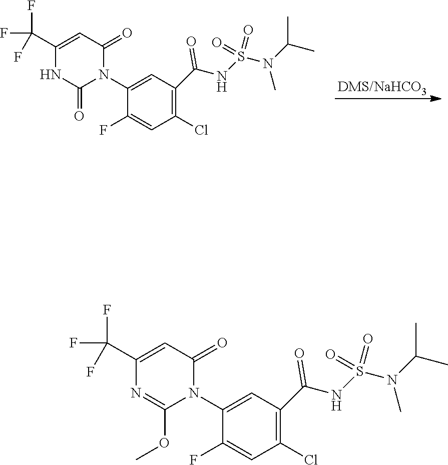

Some of the intermediates are converted to Saflufenacil in the following manner:

An isomerization reaction towards Saflufenacil using N,N′-dimethylaminopyridine (DMAP):

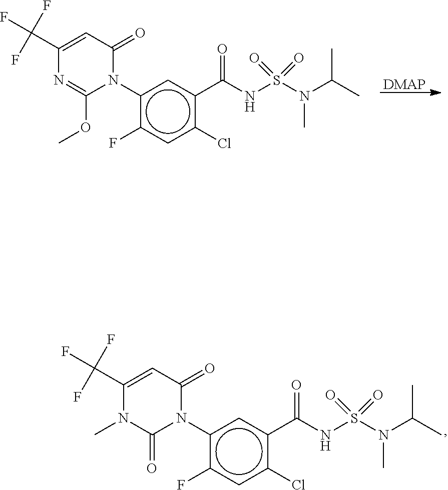

and for another example, a cyclization reaction using ethyl 4,4,4-trifluoro-3-oxobutanoate:

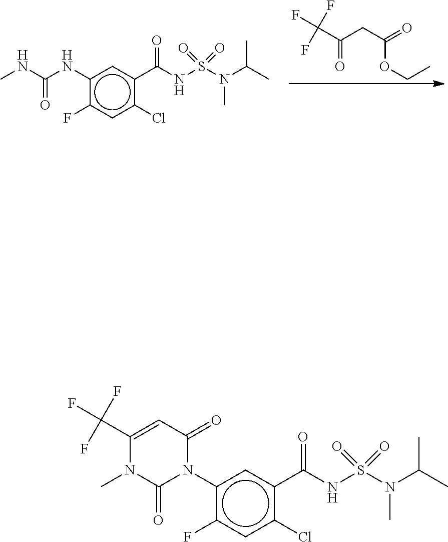

It is suspected that, surprisingly, an unexpected effect of some of the reported intermediates of the present invention of Saflufenacil synthesis, might have a pesticidal activity. Moreover, it is suspected that said intermediates are herbicides.

### Definitions

Unless defined otherwise, all technical and scientific terms used herein have the meaning commonly understood by persons of ordinary skill in the art to which this subject matter pertains.

The term “alkyl” as used herein, refers to a branched, unbranched, or cyclic carbon chain, including methyl, ethyl, propyl, isopropyl, cyclopropyl and the like.

As used herein, the term “carbonyl precursor” is a reagent used for introducing a carbonyl moiety into the molecule.

As used herein, the term “cyclization reagent” is a reagent with the following moiety:

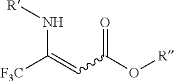

or KNCO/acetic acid
as described and exemplified in the description.

As used herein, the term “coupling reagent” is a reagent used in a condensation reactions to bind two molecules into one.

As used herein, the term “methylation reagent” is a reagent used in alkylation reactions which introduces a methyl to the molecule.

The term “a” or “an” as used herein includes the singular and the plural, unless specifically stated otherwise. Therefore, the terms “a,” “an,” or “at least one” are used interchangeably in this application.

Throughout the application, descriptions of various embodiments are described using the term “comprising”; however, it will be understood by one of skill in the art, that in some specific instances, an embodiment can be described using the language “consisting essentially of” or “consisting of.”

The term “about” herein specifically includes ±10% from the indicated values in the range. In addition, the endpoints of all ranges directed to the same component or property herein are inclusive of the endpoints, are independently combinable, and include all intermediate points and ranges.

It is understood that where a parameter range is provided, all integers within that range, and tenths thereof, are also provided by the invention as if the integers and tenths thereof are expressly described herein. For example, “0.1% to 70%” includes 0.1%, 0.2%, 0.3%, 0.4%, 0.5% etc. up to 70%.

All publications, patents and patent applications mentioned in this specification are herein incorporated in their entirety by reference into the specification, to the same extent as if each individual publication, patent or patent application was specifically and individually indicated to be incorporated herein by reference.

The following examples illustrate the practice of the present subject matter in some of its embodiments but should not be construed as limiting the scope of the present subject matter. Other embodiments apparent to persons of ordinary skill in the art from consideration of the specification and examples herein that fall within the spirit and scope of the appended claims are part of this invention. The specification, including the examples, is intended to be exemplary only, without limiting the scope and spirit of the invention.

Each embodiment disclosed herein is contemplated as being applicable to each of the other disclosed embodiments. Thus, all combinations of the various elements described herein are within the scope of the invention. In addition, the elements recited in process embodiments can be used in combination with compound embodiments described herein and vice versa.

This invention will be better understood by reference to the Examples which follow, but those skilled in the art will readily appreciate that the specific experiments detailed are only illustrative of the invention as described more fully in the claims which follow thereafter.

The invention is illustrated by the following examples without limiting it thereby.

### EXAMPLES

### Example 1. Ethyl 2-chloro-4-fluoro-5-nitrobenzoate

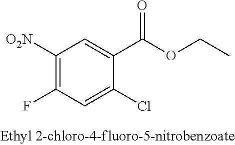 [SMILES: CCOC(=O)c1cc([N+](=O)[O-])c(F)cc1Cl]

To a clean, dry 4 necks RBF (2000 mL) equipped with a mechanical stirrer, thermocouple, condenser and additional funnel were charged 500 mL ethanol at 25-30° C., 100 g of 2-chloro-4-fluoro-5-nitrobenzoic acid and mixture was stirred for 10-15 mins up to the getting clear solution. Reaction mass was cooled to 0-5° C. and 108.6 g of Thionyl chloride (2.0 eq) was fed to the reaction mass at 0-5° C. during about 30 mins. After that the reaction mass was slowly heated to 65-70° C. and stirred at this temperature with mild reflux of solvent during 6-8 hrs at 65-70° C. Reaction was monitored by HPLC area % analysis up to residual concentration of 2-chloro-4-fluoro-5-nitrobenzoic acid less than 1%. After the reaction was finished about 400 mL of ethanol were distilled out at 60-65° C. under reduced pressure. The reaction mass was cooled to 20-25° C. and 500 ml of water were added to the reaction mass over the period of 15-20 mins at 20-25° C. After that 500 mL of isopropyl acetate were added at once to the reaction mass and the mixture was stirred for 15-20 mins. The layers were separated at 25-30° C. Top isopropyl acetate layer contains the product. Solution in isopropyl acetate was washed with 100 mL of 2% sodium bicarbonate aqueous solution. After phases separation isopropyl acetate solution of ethyl 2-chloro-4-fluoro-5-nitrobenzoate may be delivered to the next step (hydrogenation of the nitro-group) without additional purification and/or product separation. Yield of ethyl 2-chloro-4-fluoro-5-nitrobenzoate 98%.

### Example 2. Ethyl 5-amino-2-chloro-4-fluorobenzoate

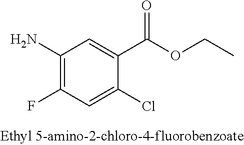

Isopropyl acetate solution of ethyl 2-chloro-4-fluoro-5-nitrobenzoate from Example 1 was introduced to the clean and dry pressure reactor equipped with mechanical stirrer, manometer and thermocouple at 25-30° C. To the solution 15 g of Raney Ni were added under nitrogen atmosphere at the same temperature. Reactor was closed and hydrogen gas pressure was applied to 90-100 PSI at 25-30° C. The mixture was stirred at the same pressure of hydrogen for 26-30 hrs at 25-30° C. up to the concentration both of starting material and hydroxylamine intermediate were reduced below 1 area % according HPLC. At the end of reaction catalyst was filtered from reaction mass through celite bed under nitrogen atmosphere at 25-30° C. The bed was washed with 100 mL of isopropyl acetate at 25-30° C. To the combined Isopropyl acetate solution were added 200 mL of Isopropyl acetate and the same volume of the solvent was distilled out at 80-85° C. under atmospheric pressure to dry reaction mixture up to the moisture content level 0.5% by KF analysis. The reaction mass was cooled to 25-30° C. and analyzed. Ethyl 5-amino-2-chloro-4-fluorobenzoate in isopropyl acetate solution may be delivered to the next step (carbamate preparation) without additional purification and/or product separation. Yield of ethyl 5-amino-2-chloro-4-fluorobenzoate 95%.

### Example 3. Ethyl 2-chloro-5-ethoxycarbonylamino-4-fluorobenzoate

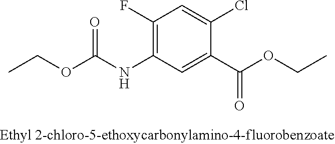 [SMILES: CCOC(=O)Nc1cc(C(=O)OCC)c(Cl)cc1F]

To the solution of ethyl 5-amino-2-chloro-4-fluorobenzoate in isopropyl acetate from Example 2 102.5 g of N,N-diethyl-aniline were added at 25-30° C. To this mixture 74 g of ethyl chloroformate were fed dropwise over the period of 15-20 mins at 25-30° C. Reaction mass was heated to 40-45° C. and maintained at this temperature for 6-8 hrs up to the reduction of starting material concentration below 1 area % by HPLC. Towards the end of reaction solid precipitation was observed. The reaction mass was cooled to 25-30° C. and 300 mL of 10% HCl were added at this temperature. The reaction mass was stirred at 25-30° C. for 30-40 mins and after that two layers were separated. Aqueous layer was sent for recovery of N,N-diethylaniline. Upper organic layer was washed with 100 mL 5% aqueous sodium bicarbonate solution and analyzed. Ethyl 2-chloro-5-ethoxycarbonylamino-4-fluorobenzoate in isopropyl acetate solution may be delivered to the next step (cyclization) without additional purification and/or product separation. Yield of ethyl 2-chloro-5-ethoxycarbonylamino-4-fluorobenzoate 97%.

### Example 4. Ethyl 2-chloro-5-(2,6-dioxo-4-(trifluoromethyl)-3,6-dihydropyrimidin-1(2H)-yl)-4-fluorobenzoate

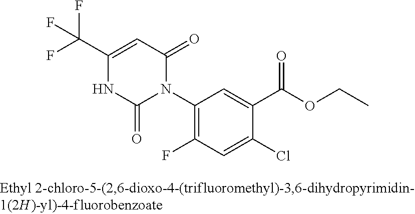 [SMILES: CCOC(=O)c1cc(-n2c(=O)cc(C(F)(F)F)[nH]c2=O)c(F)cc1Cl]

Isopropyl acetate solution of ethyl 2-chloro-5-ethoxycarbonylamino-4-fluorobenzoate from Example 3 was charged in to clean and dry RBF and most of the isopropyl acetate (about 300-350 mL) was distilled out at 55-60° C. at 600-625 mbar. To the residue were added 300 mL of N,N-dimethylacetamide and distillation of isopropyl acetate was continued at the same conditions. Toward the end of distillation to the mixture were added 200 mL of toluene and the mixture was dried by azeotropic distillation of the toluene at 60-70° C. under the vacuum. Residual water content must be not more than 0.5% by KF. To the dry solution of ethyl 2-chloro-5-ethoxycarbonylamino-4-fluorobenzoate in N,N-dimethylacetamide 104.4 g of 1,8-diazabicyclo(5.4.0)undec-7-ene (DBU) and 100.8 g of ethyl 3-amino-4,4,4-trifluorobut-2-enoate were added at 25-30° C. The reaction mass was heated to 58-62° C. under nitrogen stream for better removal of ethanol formed in the reaction. The reaction mass was stirred at these conditions for 12-14 hrs, so, that concentration of ethyl 2-chloro-5-ethoxycarbonylamino-4-fluorobenzoate was reduced below 2 area % by HPLC. The reaction mass was cooled to 25-30° C. and poured to 500 mL of 10% aqueous HCl at the temperature 10-15° C. The temperature was raised 4-5° C. and with stirring the reaction mass was warmed to 25-30° C. To the mixture 1000 mL of isopropyl acetate were added and stirring continued for 30-40 mins at 25-30° C. The layers were separated at 25-30° C. Isopropyl acetate contains product. Ethyl 2-chloro-5-(2,6-dioxo-4-(trifluoromethyl)-3,6-dihydropyrimidin-1(2H)-yl)-4-fluorobenzoate in isopropyl acetate solution may be delivered to the next step (hydrolysis) without additional purification and/or product separation. Yield of ethyl 2-chloro-5-(2,6-dioxo-4-(trifluoromethyl)-3,6-dihydropyrimidin-1(2H)-yl)-4-fluorobenzoate 85%.

### Example 5. 2-Chloro-5-(2,6-dioxo-4-(trifluoromethyl)-3,6-dihydropyrimidin-1(2H)-y)-4-fluorobenzoic Acid

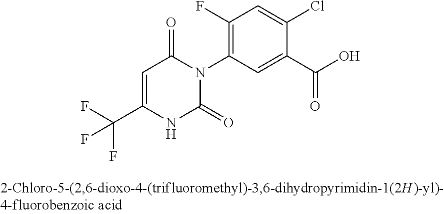 [SMILES: O=C(O)c1cc(-n2c(=O)cc(C(F)(F)F)[nH]c2=O)c(F)cc1Cl]

Ethyl 2-chloro-5-(2,6-dioxo-4-(trifluoromethyl)-3,6-dihydropyrimidin-1(2H)-yl)-4-fluorobenzoate in isopropyl acetate solution from Example 4 was delivered in to cleaned RBF at 25-30° C. and about 850 mL of the isopropyl acetate were distilled out at 45-50° C. under reduced pressure. To the mixture 600 mL of Dioxane were added and about 100 mL of Dioxane together with the rest of isopropyl acetate were distilled out under vacuum at 55-60° C. To the reaction mass 1000 mL of concentrated HCl were slowly fed at 25-30° C. The reaction mass was heated to 90-95° C. and stirred at this condition for 22-24 hrs up to reduction of ethyl 2-chloro-5-(2,6-dioxo-4-(trifluoromethyl)-3,6-dihydropyrimidin-1(2H)-yl)-4-fluorobenzoate concentration below 2 area % by HPLC. At the end of reaction Dioxane was distilled out at 55-60° C. under reduced pressure. The reaction mass was cooled to 25-30° C. and 700 mL of water were added at the same temperature. Stirring was continued for 4-6 hrs at 20-25° C. Reaction product was filtered at 25-30° C. and washed with 400 mL of water at 25-30° C. 2-Chloro-5-(2,6-dioxo-4-(trifluoromethyl)-3,6-dihydropyrimidin-1(2H)-yl)-4-fluorobenzoic acid was prepared with the purity not less than 96 area % by HPLC. Wet compound was dispersed in 500 mL of Toluene and mixture was dried by azeotropic distillation at 100-110° C. up to moisture content in reaction mass not more than 0.5% by KF. The reaction mass was cooled to 20-25° C. and stirred at this temperature during 3-4 hrs. 2-Chloro-5-(2,6-dioxo-4-(trifluoromethyl)-3,6-dihydropyrimidin-1(2H)-yl)-4-fluorobenzoic acid was filtered at 20-25° C. and dried under reduced pressure at 45-50° C. 108 g of the product was separated with assay 97%. Yield 86% on the hydrolysis step or 66% on five telescopic steps.

### Example 6. 2-Chloro-5-(2,6-dioxo-4-(trifluoromethyl)-3,6-dihydropyrimidin-1(2H)-yl)-4-fluoro-N-(N-isopropyl-N-methylsulfamoyl)benzamide

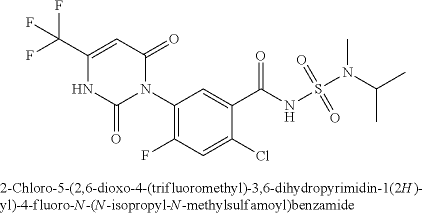 [SMILES: CC(C)N(C)S(=O)(=O)NC(=O)c1cc(-n2c(=O)cc(C(F)(F)F)[nH]c2=O)c(F)cc1Cl]

To a clean, dry 4 necks RBF equipped with a mechanical stirrer, thermocouple, condenser and additional funnel were charged 40 ml of acetonitrile, 13 g of N-isopropyl-N-methylsulfamoylamide and 15.75 g of potassium carbonate at 25-30° C. and under nitrogen atmosphere. The reaction mass was heated to attain 55-60° C. Into another RBF were charged 100 mL of acetonitrile and under nitrogen atmosphere was added 20 g of 2-chloro-5-(2,6-dioxo-4-(trifluoromethyl)-3,6-dihydropyrimidin-1(2H)-yl)-4-fluorobenzoic acid and 12 g of 1,1′-carbonyldiimidizole (CDI) at 25-30° C. The reaction mass was heated to 55-60° C. and stirred at this temperature during about 1.5 h to produce 3-(4-chloro-2-fluoro-5-(1H-imidazole-1-carbonyl)phenyl)-6-(trifluoromethyl)pyrimidine-2,4(1H,3H)-dione. Residual concentration of 2-chloro-5-(2,6-dioxo-4-(trifluoromethyl)-3,6-dihydropyrimidin-1(2H)-yl)-4-fluorobenzoic acid was below 2 area % by HPLC.

A prepared solution of 3-(4-chloro-2-fluoro-5-(1H-imidazole-1-carbonyl) phenyl)-6-(trifluoromethyl)pyrimidine-2,4(1H,3H)-dione was fed to the mixture of N-isopropyl-N-methylsulfamoylamide and potassium carbonate over the period of 10-15 mins at 55-60° C. The reaction mass was stirred at 55-60° C. for 6-8 hrs up to the moment that concentration of 3-(4-chloro-2-fluoro-5-(1H-imidazole-1-carbonyl)phenyl)-6-(trifluoromethyl)pyrimidine-2,4(1H,3H)-dione was not more than 2 area % by HPLC. With the end of reaction, the mixture was cooled to 25-30° C. and stirred at this temperature for 25-30 mins. K₂CO₃ was filtered from the reaction mass at 25-30° C. and washed with 20 mL of acetonitrile. The filtrate (contains the product) was charged into clean RBF and heated to 40-45° C. About 80 mL of acetonitrile was distilled from the filtrate at 40-45° C. under reduced pressure (650 mbar). The reaction mass was cooled to 25-30° C. and 200 mL of 2-methyl-THF and 100 mL of water were added at once. With good stirring the reaction mass was cooled to 0-5° C. and the pH of reaction mass was adjusted to 1-2 with concentrated HCl (about 25 mL) at the same temperature. Cooling and stirring were stopped and layers were separated at 25-30° C. Top organic layer contained the product. Bottom aqueous layer contained imidazole hydrochloride. Top organic layer was charged to the clean RBF and 60 mL of water were added at 25-30° C. With good stirring the pH of the aqueous phase was adjusted to 5.8-6.0 with 5% aqueous sodium bicarbonate. The layers were separated at 25-30° C. Top organic layer contained the product. Bottom aqueous layer contained sodium salt of 2-chloro-5-(2,6-dioxo-4-(trifluoromethyl)-3,6-dihydropyrimidin-1(2H)-yl)-4-fluorobenzoic acid. Top organic layer was charged into clean RBF and about 160 mL of 2-methyl THF were distilled out at 40-45° C. under reduced pressure. To the residue 60 mL of toluene were added and additional about 30 mL of the solvent (mainly rest of 2-methyl THF) were distilled out at the same conditions. Reaction mass was cooled and stirred for 1-2 hrs at 25-30° C. Precipitated solid was filtered and washed with 20 Ml of toluene at 25-30° C. Wet solid was charged into clean RBF and 40 ml of acetone were added at 25-30° C. The mixture was stirred for 30 mins at 25-30° C. and 100 ml of water were fed slowly at the same temperature. The mixture was stirred during 4 hrs for full crystallization of the product and the solid compound was filtered at 25-30° C. After drying at 50-55° C. under reduced pressure for 10-12 hrs 19 g of 2-chloro-5-(2,6-dioxo-4-(trifluoromethyl)-3,6-dihydropyrimidin-1(2H)-yl)-4-fluoro-N-(N-isopropyl-N-methylsulfamoyl)benzamide were prepared. Yield 70%.

The synthesis of the starting materials and Saflufenacil may be carried on by using prior art examples, of instance of the WO 2001/083459 publication.

1. A process for preparing compounds of the Formula I: 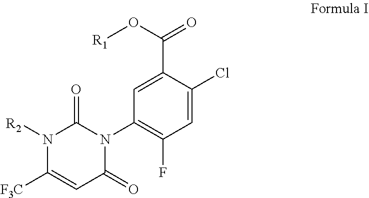
wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl;
the process comprising the following steps:
a) a reaction of the aniline moiety of the compound of Formula II: 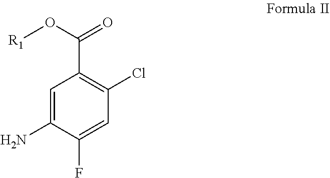
wherein R₁ is hydrogen with a carbonyl precursor, wherein the carbonyl precursor is ethyl 4,4,4-trifluoroacetoacetate, to obtain the compound of Formula II′: 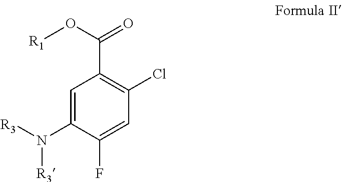
wherein R₁ is hydrogen;
R₃ is hydrogen;
R₃′ is 4,4,4-trifluoroacetoacetate; and
b) a reaction of the resulting compound of Formula II′ with a cyclization reagent which is KOCN in AcOH;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is phosgene, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ and R₃′ together are carbonyl; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
a reaction of the aniline moiety of the compound of Formula II wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents, with a carbonyl precursor, wherein the carbonyl precursor is ethyl chloroformate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl formate; and
b) a reaction of the resulting compound of Formula II′ with a cyclization reagent which is ethyl 3-amino-4,4,4-trifluorocrotonate or ethyl 3-methylamino-4,4,4-trifluorocrotonate, followed by a cyclization reaction;
or
wherein R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents with a carbonyl precursor, wherein the carbonyl precursor is ethyl 4,4,4-trifluoroacetoacetate, to obtain the compound of Formula II′, wherein
R₁ is selected from a group consisting of C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents;
R₃ is hydrogen;
R₃ is ethyl 4,4,4-trifluoroacetoacetate; and
b) a reaction of the resulting compound of Formula II′ with a cyclization reagent which is KOCN in AcOH.

2. A process according to claim 1, wherein the carbonyl precursor comprises: phosgene, ethyl 4,4,4-trifluoro-3-oxobutanoate, ethyl chloroformate.

3. A process according to claim 1, wherein a cyclization reagent comprises: potassium cyanate in acetic acid, alkyl 3-amino-4,4,4-trifluorobut-2-enoate or alkyl (3-methylamino-4,4,4-trifluorobut-2-enoate.

4. A process according to claim 1, wherein the process is carried out in an aprotic organic solvent selected from a group consisting of: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

5. A process according to claim 1, wherein the process of step a) is carried out at a temperature of 0° C. to 150° C.

6. A process according to claim 1, wherein the process of step b) is carried out at a temperature of 20° C. to 100° C.

7. A process according to claim 1, comprising a step of a hydrolysis reaction of the compounds of Formula I where R₁ is not hydrogen using an acidic or basic catalysis, to obtain a compound of Formula III: 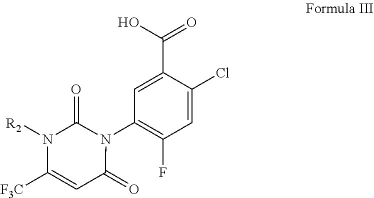
wherein
R₂ is a hydrogen or methyl.

8. A process according to any of claims 1 to 7, comprises the step of condensation reaction of an acid of Formula III 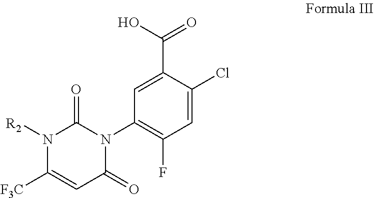
wherein
R₂ is a hydrogen or methyl; and a compound of Formula 2, 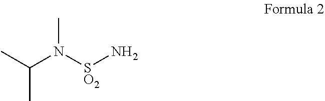
using a coupling reagent, to obtain the compound of Formula IV:

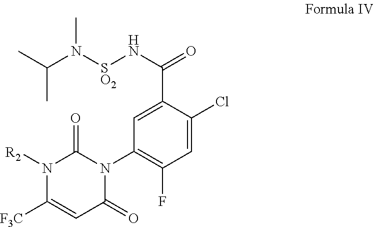

9. A process according to claim 8, wherein the coupling reagent is selected from a list comprising: halogenated reagents like oxalyl chloride, thionyl chloride, phosgene, Vilsmeier reagents; CDI, carbon diimides, HBTU.

10. A process according to claim 8, wherein the reaction is carried out in a solvent selected from a group comprising: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

11. A process according to claim 8, wherein reaction is carried out in a temperature of 0° C. to 100° C.

12. A process according to any of claims 1 to 11, comprises an additional step of methylation reaction on the compound of the Formula IV, when R₂ is hydrogen, using a methylation reagent, to obtain saflufenacil: 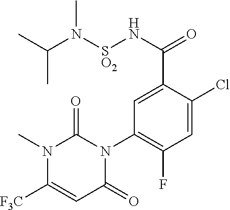 [SMILES: CC(C)N(C)S(=O)(=O)NC(=O)c1cc(-n2c(=O)cc(C(F)(F)F)n(C)c2=O)c(F)cc1Cl]
saflufenacil.

13. A process according to claim 12, wherein the methylation reagent is selected from a list comprising: dimethyl sulfate, methyl bromide or methyl iodide.

14. A process according to claim 12, wherein the reaction is carried out in a solvent selected from a group comprising: MeCN, DMF, dimethylacetamide, NMP, DMSO, ethylene or propylene carbonate, ethers such as 1,4-dioxane, MTBE, MCPE, Me-THF or THF, esters like ethyl acetate, iso-propyl acetate and aromatic compounds selected from a group comprising toluene and chlorobenzene.

15. A process according to claim 12, wherein reaction is carried out in a temperature of 0° C. to 100° C.

16. A compound of the general Formula I: 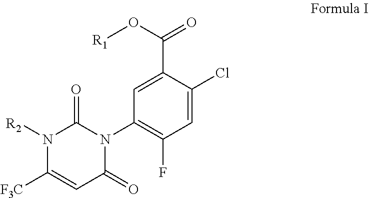
wherein
R₁ is a hydrogen, C₁₋₁₂ straight or branched alkyl, which may be substituted with 1 or more substituents, a C₃₋₁₀ cycloalkyl, which may be substituted with 1 or more substituents, a C₆₋₁₀ aromatic ring which may be substituted with 1 or more substituents, a C₅₋₁₀ heteroaromatic ring, which may be substituted with 1 or more substituents; and
R₂ is a hydrogen or methyl.

17. N-[Methyl(isopropyl)aminosulfonyl]-2-chloro-4-fluoro-5-(3-methylureido)benzamide.

18. 4-fluoro-N-(N-isopropyl-N-methylsulfamoyl)-2-methoxy-5-(3-methyl-2,6-dioxo-4-(trifluoromethyl)-3,6-dihydropyrimidin-1(2H)-yl) benzamide.

19. 3-(5-[N-Diethylaminosulfonylaminocarbonyl]-4-chloro-2-fluorophenyl)-2,4-dioxo-1-methyl-6-(trifluoromethyl)-1,2,3,4-tetrahydropyrimidine.

20. 3-[5-(N-Methyl[N-methyl(isopropyl)aminosulfonyl]aminocarbonyl)-4-chloro-2-fluorophenyl]-1-methyl-2,4-dioxo-6-(trifluoromethyl)-1,2,3,4-tetrahydropyrimidine.

21. 3-(5-[N-Methyl(isopropyl)aminosulfonylaminocarbonyl]-4-chloro-2-fluorophenyl)-2-methoxy-4-oxo-6-(trifluoromethyl)-3,4-dihydropyrimidine.
